(19) United States
(12) Patent Application Publication
(10) Pub. No.: US 2015/0025103 A1
(43) Pub. Date: Jan. 22, 2015

(54) ORAL LIQUID CONCENTRATE COMPRISING BROMPHENIRAMINE, PSEUDOEPHEDRINE AND DEXTROMETHORPHAN

(71) Applicant: WOCKHARDT LIMITED, Aurangabad (IN)

(72) Inventors: Mayur Bhamare, Dhule (IN); Rajendra Nagore, Aurangabad (IN); Girish Kumar Jain, Delhi (IN)

(73) Assignee: WOCHKARDT LIMITED

(21) Appl. No.: 14/363,794

(22) PCT Filed: Nov. 1, 2012
(86) PCT No.: PCT/IB2012/056086

§ 371 Date: Jun. 7, 2014

(30) Foreign Application Priority Data
Dec. 9, 2011 (IN) 3482/MUM/2011

(51) Int. Cl. A61K 47/26; A61K 31/137; A61K 47/14; A61K 47/10; A61K 47/12; A61K 31/4402; A61K 31/485

(52) U.S. Cl. CPC A61K 47/26; A61K 31/4402; A61K 31/137; A61K 31/485; A61K 47/10; A61K 47/12; A61K 47/14
USPC 514/289

(57) ABSTRACT

There is provided a liquid oral concentrate comprising combination of dextromethorphan, bromrpheniramine and pseudoephedrine or pharmaceutically acceptable salts thereof. The invention further provides process for preparation of such compositions.

### FIELD OF THE INVENTION

The present invention relates to a taste masked ready-to-use liquid pharmaceutical concentrate composition for oral administration comprising more than 0.04% w/v of brompheniramine, 0.60% w/v of pseudoephedrine and 0.20% w/v of dextromethorphan or pharmaceutically acceptable salts thereof and which is used for treating symptoms of the common cold and allergic rhinitis. The invention further provides process for preparation of such compositions.

### BACKGROUND OF THE INVENTION

Upper respiratory symptoms include nasal congestion, sinusitis, cough, cold, cold-like symptoms, allergic rhinitis resulting from a cold or influenza infection or allergic reactions, upper respiratory mucosal congestions such as those seen in perennial and allergic rhinitis. Eustachian tube congestion, runny nose, post nasal drip are the most common ailments which are frequently seen in individuals. Though the ailments generally are not life threatening, it may result in severe discomfort and hamper day-to-day life of the individuals.

The symptoms are treated using variety of therapeutic agents such as antihistamines, decongestants, cough suppressants or antitussives, expectorants and preferably combinations thereof. Several attempts have been made to develop compositions comprising combination of the said therapeutic active agents in different dosage forms.

Some of the commercially available antihistamine drugs are Loratadine (Claritin, Tavist), Brompheniramine (Dimetane), Chlorpheniramine (Chlor-Trimeton), Diphenhydramine (Benadryl), Cetirizine (Zyrtec).

Some of the commercially available Decongestants are pseudoephedrine (Drixoral Non-Drowsy, Sudafed Nasal Decongestant, Children's Dimetapp Decongestant Infant phenylpropanolamine (Acutrim 16 Hour, Acutrim II, Maximum Strength, Acutrim Late Day) phenylephrine (Dimetapp Toddler's Drops Decongestant).

Some of the commercially available antitussives are carbetapentane (Solotuss), Benzonatate (Zonatuss, TessalonPerles, Tessalon), Dextromethorphan (Benylin DM, Creo-Terpin).

Dextromethorphan is marketed as dextromethorphan hydrobromide and dextromethorphan polistirex. Chemically dextromethorphan hydrobromide is a salt of the methyl ether of the dextrorotatory isomer of levorphanol. It is chemically designated as 3-methoxy-17-methyl-9α, 13α, 14α-morphinan hydrobromide monohydrate with the following structural formula I:

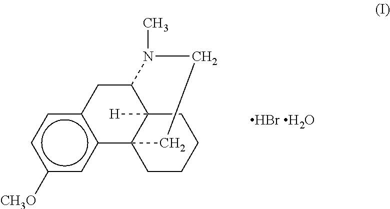

Dextromethorphan polistirex (dextromethorphan hydrobromide complexed with resin) is marketed under the trade name Delsym® by Reckitt Benckiser in the form of extended release suspension indicated for the treatment of non-productive cough.

Pseudoephedrine is marketed as pseudoephedrine hydrochloride and pseudoephedrine sulfate. Pseudoephedrine hydrochloride, is chemically [S-(R*,R*)]-α-[1-(methylamino)ethyl]-benzenemethanol hydrochloride having the structural formula (II):

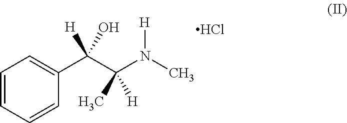

Pseudoephedrine hydrochloride is marketed as extended release tablets under the trade name “Sudafed 24 Hour®” by Alza and indicated for nasal and sinus congestion. Pseudoephedrine is available in different dosage forms including tablet, extended release tablet, capsule and suspension as a decongestant medication.

Brompheniramine was marketed as Brompheniramine maleate under the trade name DIMETANE-DX® in the form of syrup by Robins AS and as DIMETANE extended release tablets marketed by Wyeth. Chemically Brompheniramine is γ-(4-Bromophenyl)-N,N-dimethyl-2-pyridinepropanamine with the structural formula (III).

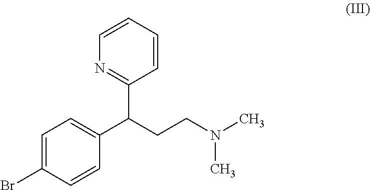 [SMILES: CN(C)CCC(c1ccc(Br)cc1)c1ccccn1]

Liquid formulations for oral delivery of pharmaceutical agents are desirable because certain patients, such as children and the elderly, are unable to swallow capsules or tablets.

Liquid formulations comprising dextromethorphan, brompheniramine and pseudoephedrine are available over-the-counter under the brand name Bromfed DM and Dimetane DX. Each 5 ml of Bromfed DM contains 2 mg Brompheniramine maleate, 30 mg pseudoephedrine hydrochloride, 10 mg dextromethorphan Hydrobromide and alcohol. Bromfed DM is indicated for the treatment of the symptoms of the common cold and allergic rhinitis, such as runny or stuffy nose, cough, itchy or watery eyes and sneezing. The dosing schedule includes administration of 2 teaspoonfuls every 4 hours i.e. 10 ml of the syrup has to be administered every 4 hours.

Dimetane DX is another liquid product of brompheniramine maleate, pseudoephedrine hydrochloride, and dextromethorphan Hydrobromide. Each 5 ml of Dimetane DX contains 2 mg brompheniramine maleate, 30 mg pseudoephedrine hydrochloride, 10 mg dextromethorphan Hydrobromide. As per the dosing schedule of Dimetane DX two teaspoonfuls has to be administered every 4 to 6 hours. The total dose should not exceed 12 teaspoonfuls in a 24 hours period.

Thus, a total of 60 ml of Bromfed or Dimetane DX is administered to a patient in a day, which is large volume to be swallowed. This often leads to non-compliance of patient to the treatment. Thus, there is a need for the development of an oral liquid concentrate thereby decreasing the total amount of liquid to be administered to a patient.

Several concentrate-based products are available in the market. For instance, Navane® Concentrate, Sinequan® Concentrate, & Trilafon® Concentrate. These concentrates either requires alcohol as a solubilizer or when alcohol is not used as solubilizer the concentrate needs to be diluted before administration. Thus, alcohol is usually used as a solvent for solubilizing and preparing an oral concentrate of the active mixture. Such compositions suffer from severe drawback of instability due to evaporation of a low boiling solvent like alcohol. This is particularly true as the products are used in home environment, which cannot be precisely controlled with respect to temperature, which ultimately may hamper product stability.

Further, brompheniramine, pseudoephedrine and dextromethorphan are all bitter and unpleasant tasting drugs. Dextromethorphan has along with bitter taste an un-aesthetic mouth-feel and an unpleasant after-taste. In order to ensure better patient compliance bitterness masking becomes essential.

Taste masking is usually achieved by use of sugar base or sugar solutions. Use of sugar syrups in pharmaceutical composition often leads to microbial contamination leading to instability of composition on storage. Further, the sugar syrups have high caloric values, which is undesirable for diabetic or obese patients.

Several attempts have been made to provide improved compositions comprising dextromethorphan, pseudoephedrine and brompheniramine.

U.S. Pat. No. 5,196,436 discloses antitussive pharmaceutical compositions for the peroral administration of dextromethorphan.

U.S. Pat. No. 6,869,618 discloses a manufacturing process for the preparation of liquid or semi-solid dosage forms containing a tannate salt complex of active pharmaceutical ingredients.

U.S. Pat. No. 7,101,572 discloses a substantially taste masked aqueous liquid pharmaceutical composition that contains an otherwise unpleasant tasting drug.

U.S. Pat. No. 4,996,047 discloses oral controlled-release pharmaceutical preparations comprising drug-ion-exchange resin complex.

U.S. Pat. No. 6,509,492 discloses liquid suspension comprising pseudoephedrine tannate, chlorpheniramine tannate and dextromethorphan tannate.

U.S. Pat. No. 6,790,980 discloses pharmaceutical liquid suspension of tannate therapeutic agents such as dexchlorpheniramine, chlorpheniramine, pseudoephedrine, dextromethorphan.

U.S. Pat. No. 5,980,882 discloses a pharmaceutical composition comprising a drug-resin complex and a chelating agent.

U.S. Pat. No. 7,094,429 discloses a process of preparing tannate salt complex of an antihistamine, a decongestant, an antitussive or anticholinergic.

U.S. Pat. No. 5,196,436 discloses antitussive composition for peroral administration consisting dextromethorphan and orally-acceptable pharmaceutical carrier in the form of an aqueous-based liquid, or solid dissolvable in the mouth.

U.S. Patent Application No. 20060121066 discloses a pharmaceutical composition comprising sucralose to mask a bitter taste of any active ingredients.

US Application 20050232993A1 discloses pharmaceutical dosage form comprising an antihistaminic drug and one second drug selected from decongestants, antitussives, expectorants, mucus thinning drugs, analgesics and antihistamines, both having different plasma half-lives.

In spite of the several attempts made in the art for preparing compositions comprising combination of therapeutic agents, there still exists a continuing need of a taste masked, alcohol free, ready to use oral liquid concentrate comprising dextromethorphan, brompheniramine and pseudoephedrine and administration of which may minimize the occurrence of adverse events and improve patient compliance, thus encouraging patient's adherence to the prescribed dosing regimen. An ideal composition should have good tasting presentation to achieve higher patient compliance.

Preparing the concentrate containing mixture of such bitter drugs, however, would impose more likelihood of a bitterer product. Thus, there is a need to develop a sugar free ready to use oral liquid concentrate.

The compositions of the present invention are alcohol free, thus are advantageous in terms of being non-addictive and abuse resistant.

An oral sugar free ready-to-use liquid concentrate of present invention comprising fixed dose combination of dextromethorphan, brompheniramine and pseudoephedrine is stable and has acceptable taste, thus offers a significant improvement to the existing formulations, providing better and greater choice for both the prescriber and the patient. This is of importance with regard to the issue of non-compliance with treatment, which is believed to affect up to 50% of outpatients and appears to be a particular problem with elderly, pediatric and psychiatric patients (B. Blackwell, Drug Therapy: Patient Compliance, New. Eng. J. Med. 1973, 289(5):249 52).

### SUMMARY OF THE INVENTION

In one general aspect of the invention, there is provided an oral ready-to-use pharmaceutical liquid composition comprising more than 0.04% w/v of brompheniramine, 0.60% w/v of pseudoephedrine and 0.20% w/v of dextromethorphan or pharmaceutically acceptable salts thereof.

In another general aspect of the invention, there is provided an oral ready-to-use pharmaceutical liquid composition comprising more than 0.04% w/v of brompheniramine, 0.60% w/v of pseudoephedrine and 0.20% w/v of dextromethorphan or pharmaceutically acceptable salts thereof, wherein the composition is free of alcohol.

In another general aspect of the invention, there is provided an oral ready-to-use pharmaceutical liquid composition comprising more than 0.04% w/v of brompheniramine, 0.60% w/v of pseudoephedrine and 0.20% w/v of dextromethorphan or pharmaceutically acceptable salts thereof, wherein the composition is free of sugar.

In another general aspect of the invention, there is provided an oral taste masked ready-to-use pharmaceutical liquid composition comprising more than 0.04% w/v of brompheniramine, 0.60% w/v of pseudoephedrine and 0.20% w/v of dextromethorphan or pharmaceutically acceptable salts thereof.

In another general aspect of the invention, there is provided an oral ready-to-use liquid concentrate comprising more than 0.04% w/v of brompheniramine, 0.60% w/v of pseudoephedrine and 0.20% w/v of dextromethorphan or pharmaceutically acceptable salts thereof.

In another general aspect of the invention, the volume of the unit doses of the composition is less than 5 ml.

In another general aspect of the invention, each 5 ml of the liquid composition comprises more that 10 mg of dextromethorphan hydrobromide, more than 2 mg of brompheniramine maleate and more than 30 mg of pseudoephedrine hydrochloride along with pharmaceutically acceptable excipients.

In another general aspect of the invention, there is provided an oral ready-to-use pharmaceutical liquid composition comprising about 10 mg of dextromethorphan, about 2 mg of brompheniramine and about 30 mg of pseudoephedrine or pharmaceutically acceptable salts thereof in each 4 ml of the liquid composition along with one or more pharmaceutically acceptable excipients.

In another general aspect of the invention, there is provided a stable oral ready-to-use pharmaceutical liquid composition comprising more than 0.04% w/v of brompheniramine, 0.60% w/v of pseudoephedrine and 0.20% w/v of dextromethorphan or pharmaceutically acceptable salts thereof, characterized in that said composition retains at least 90% w/w of total potency of brompheniramine, pseudoephedrine, and dextromethorphan or pharmaceutically acceptable salts thereof after storage at 25° C. and 40% relative humidity or 25° C. and 40% relative humidity for at least 3 months.

In another general aspect of the invention, there is provided an oral ready-to-use pharmaceutical liquid composition comprising about 0.05% w/v of brompheniramine, 0.75% w/v of pseudoephedrine and 0.25% w/v of dextromethorphan or pharmaceutically acceptable salts thereof.

In another general aspect of the invention, the liquid composition is spill resistant.

In another general aspect of the invention, the liquid composition of the present invention is in the form of solution, syrup, suspension or emulsion.

In another general aspect of the invention, there is provided a method for treating symptoms of upper respiratory tract infection, common cold, or allergic rhinitis by administering an oral ready-to-use pharmaceutical liquid composition comprising more than 0.04% w/v of brompheniramine, 0.60% w/v of pseudoephedrine and 0.20% w/v of dextromethorphan or pharmaceutically acceptable salts thereof.

The pharmaceutical composition of the invention further may comprise pharmaceutically acceptable excipients wherein excipients may be selected from one or more of solvent, co-solvent, buffering agents, suspending agents, surfactants, thickening agents or viscosity modifiers, sweeteners, flavors and preservatives.

### DETAILED DESCRIPTION OF THE INVENTION

The present inventors while working on the development of liquid concentrate comprising dextromethorphan, brompheniramine and pseudoephedrine have surprisingly found that the there is no need of alcohol to solubilize the active ingredients. The aqueous based oral concentrate comprising a fixed dose combination of dextromethorphan, brompheniramine and pseudoephedrine required no dilution prior to administration.

By virtue of being a concentrate, having less volume to be swallowed, an oral liquid concentrate of the present invention not only would offer an alternative to those patients who dislike or have difficulty swallowing tablets or capsules, and would particularly suitable for pediatric and geriatric patients as less volume has to be administered to them, which can be administered more accurately by use of dose dispensers, catridages or droppers.

Further, the pharmaceutical liquid composition of the present invention is sugar free, thus can advantageously be administered to diabetic, obese and health conscious people. Further, being sugar free, the chances of microbial contamination are reduced leading to high stability during storage till use.

The oral ready-to-use pharmaceutical liquid composition of the present invention comprises more than 0.04% w/v of brompheniramine, 0.60% w/v of pseudoephedrine and 0.20% w/v of dextromethorphan or pharmaceutically acceptable salts thereof. The composition is free of alcohol.

Further, the pharmaceutical liquid composition of the present invention is in the form of a liquid concentrate.

As used herein, the term “concentrate” is intended to designate a liquid wherein relatively high amount of the solutes are dispersed or dissolved therein. For instance, a liquid concentrate of brompheniramine, pseudoephedrine, and dextromethorphan contains more than 0.04% w/v of brompheniramine, 0.60% w/v of pseudoephedrine and 0.20% w/v of dextromethorphan or pharmaceutically acceptable salts thereof.

In an embodiment, each 5 ml of the liquid concentrate comprises more that 10 mg of dextromethorphan hydrobromide, more than 2 mg of brompheniramine maleate and more than 30 mg of pseudoephedrine hydrochloride along with one or more pharmaceutically acceptable excipients.

In another embodiment, each 4 ml of the liquid concentrate comprises about 10 mg of dextromethorphan, about 2 mg of brompheniramine and about 30 mg of pseudoephedrine or pharmaceutically acceptable salts thereof along with one or more pharmaceutically acceptable excipients.

In a further embodiment, the pharmaceutical liquid concentrate comprising about 0.05% w/v of brompheniramine, 0.75% w/v of pseudoephedrine and 0.25% w/v of dextromethorphan or pharmaceutically acceptable salts thereof.

As used herein, the term “salt” refers to any pharmaceutically acceptable salt {e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts. Examples of bases include, but are not limited to, alkali metals (e.g., sodium) hydroxides, alkaline earth metals (e.g., magnesium), hydroxides, ammonia, and compounds of formula NW4⁺, wherein W is C₁₋₄ alkyl, and the like. Examples of salts include, but are not limited to: acetate, adipate, alginate, aspartate, benzoate, benzenesulfonate, bisulfate, butyrate, citrate, camphorate, camphorsulfonate, digluconate, dodecylsulfate, cyclopentanepropionate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like.

The term “dextromethorphan”, as used herein, refers to dextromethorphan base, or any pharmaceutically acceptable salt thereof. In an embodiment, invention dextromethorphan salt could be dextromethorphan hydrobromide.

The term “brompheniramine”, as used herein, refers to brompheniramine base, or any pharmaceutically acceptable salt thereof. In an embodiment, invention brompheniramine salt could be brompheniramine maleate.

The term “pseudoephedrine”, as used herein, refers to pseudoephedrine base, or any pharmaceutically acceptable salt thereof. In an embodiment, invention pseudoephedrine salt could be pseudoephedrine hydrochloride.

The term “non-alcoholic” or “free of alcohol”, as used herein, refers to the composition that comprises less than 0.01% w/v alcohol by total volume of the composition.

The term “ready-to-use”, as used herein, refers to a composition available for immediate use and requiring no dilution prior to use.

In a further embodiment, the oral taste masked ready-to-use pharmaceutical liquid composition comprises more than 0.04% w/v of brompheniramine, 0.60 % w/v of pseudoephedrine and 0.20% w/v of dextromethorphan or pharmaceutically acceptable salts thereof.

The ready-to-use pharmaceutical liquid composition of the present invention exhibits excellent storage stability and retains at least 90% w/w of total potency of brompheniramine, pseudoephedrine, and dextromethorphan or pharmaceutically acceptable salts thereof after storage at 25° C. and 40% relative humidity or 25° C. and 40% relative humidity for at least 3 months.

The ready-to-use pharmaceutical liquid composition of the invention further may comprise pharmaceutically acceptable excipients wherein excipients may be selected from one or more of solvent, co-solvent, buffering agents, suspending agents, surfactants, thickening agents or viscosity modifiers, sweeteners, flavors and preservatives.

Suitable solvents and co-solvents may include but not limited to one or more of water, sorbitol solution, glycerin, propylene glycol, polyethylene glycols, glucofurol and mixtures thereof.

Suitable buffering agents may include one or more of a bicarbonate salt of a Group IA metal, an alkali earth metal buffering agent, a calcium buffering agent, a magnesium buffering agent, an aluminum buffering agent and the like, sodium bicarbonate, potassium bicarbonate, magnesium hydroxide, magnesium lactate, magnesium gluconate, magnesium oxide, magnesium aluminate, magnesium carbonate, magnesium silicate, magnesium citrate, aluminum hydroxide, aluminum phosphate, aluminum hydroxide/magnesium carbonate, potassium carbonate, potassium citrate, aluminum hydroxide/sodium bicarbonate coprecipitate, aluminum glycinate, aluminum magnesium hydroxide, sodium citrate, sodium tartrate, sodium acetate, sodium carbonate, sodium (polyphosphate, sodium dihydrogen phosphate, potassium polyphosphate, sodium pyrophosphate, potassium pyrophosphate, disodium hydrogenphosphate, dipotassium hydrogenphosphate, trisodium phosphate, tripotassium phosphate, potassium metaphosphate, calcium acetate, calcium glycerophosphate, calcium chloride, calcium hydroxide, calcium lactate, calcium carbonate, calcium gluconate, calcium bicarbonate, calcium citrate, calcium phosphate magnesium phosphate, potassium phosphate, sodium phosphate, trihydroxymethylaminomethane, ail amino acid, an acid salt of an amino acid, an alkali salt of an amino acid, and mixtures thereof.

Suitable surfactants are those known to ordinary skilled in the art and may include one or more of amphoteric, non-ionic, cationic or anionic surfactants. Suitable surfactants comprises one or more of sodium lauryl sulfate, monooleate, monolaurate, monopalmitate, monostearate or another ester of polyoxyethylene sorbitane, sodium dioctylsulfosuccinate (DOSS), lecithin, stearylic alcohol, cetostearylic alcohol, cholesterol, polyoxyethylene ricin oil, polyoxyethylene fatty acid glycerides, poloxamer, and cremophore RH 40.

Suitable thickening agents or viscosity modifiers may include one or more of methylcellulose, carboxymethylcellulose, microcrystalline cellulose, ethylcellulose, hydroxyethylcellulose, hydroxypropylcellulose, hydroxypropylmethylcellulose, alginate, carageenan, xanthan gum, acacia, tragacanth, locust bean gum, guar gum, carboxypolymethylene, polyvinyl pyrrolidone, polyvinyl alcohol, poloxamer, magnesium aluminum silicate (veegum), bentonite, hectorite, povidone, maltitol, chitosan or mixture thereof.

Suitable sweetener may include but not limited to one or more of monosaccharides, disaccharides and polysaccharides, e.g. xylose, ribose, glucose, mannose, galactose, fructose, sucrose, maltose, invert sugar, partially hydrolyzed starch, corn syrup solids, mannitol, xylitol, D-sorbitol, erythritol, pentitol, hexitol, malitol, dihydrochalcones, monellin, steviosides or glycyrrhizin; saccharin in free acid form, soluble saccharin salts, (e.g. sodium or calcium saccharin salts, cyclamate salts or acesulfame K); L-aspartic acid derived sweeteners, (e.g. aspartame); water-soluble sweeteners derived from naturally occurring water-soluble sweeteners, (e.g. sucralose); and protein based sweeteners, (e.g. thaumatococcus danielli (Thaumatin I and II)).

Suitable flavoring agents may include those known to the skilled artisan, such as natural, “natural-like” and artificial flavors. These flavors may be chosen e.g. from synthetic flavor oils, flavoring aromatics, oleo-resins and extracts derived e.g. from plants, leaves, flowers or fruits.

Representative flavors may include one or more of spearmint oil, cinnamon oil, peppermint oil, clove oil, bay oil, thyme oil, cedar leaf oil, oil of nutmeg, oil of sage, oil of bitter almonds, vanilla, chocolate, coffee, cocoa and citrus oil, lemon, orange, cherry, grape, lime or grapefruit, and fruit essences, e.g. apple, pear, peach, strawberry, raspberry, cherry, plum, pineapple or apricot; mints such as peppermint (including menthol, especially levomenthol), and aldehydes or esters, (e.g. cinnamyl acetate, cinnamaldehyde, citral, diethylacetal, dihydrocarvyl acetate, eugenyl formate or p-methylanisol; alpha-citral (geranial) and beta-citral (neral); decanal; ethyl vanillin; piperonal (heliotropine); vanillin; alpha-amyl cinnamaldehyde; butyraldehyde; valeraldehyde; citronellal; decanal; aldehyde C-8; aldehyde C-9; aldehyde C-12; 2-ethyl butyraldehyde; hexenal, i.e. trans-2; tolyl aldehyde; veratraldehyde; 2,6-dimethyl-5-heptenal (melonal); 2-6-dimethyloctanal; 2-dodecenal and the like).

Preservatives may include but not limited to one or more of sodium benzoate, sorbates, such as potassium sorbate, salts of edetate (also known as salts of ethylenediaminetetraacetic acid or EDTA, such as disodium edetate), benzaldionium chloride, parabens and the like.

Suspending agents may include but not limited to one or more from cellulose derivatives, clays, natural gums, synthetic gums, or other agents known in the art. Specific suspending agents, by way of example, include microcrystalline cellulose, sodium carboxymethylcellulose, powdered cellulose, ethymethylcellulose, hydroyxypropyl methylcellulose, methylcellulose, ethylcellulose, ethylhydroxy ethylcellulose, hydroxypropyl cellulose, attapulgite, bentonite, hectorite, montmorillonite, silica gel, fumed silicon dioxide, colloidal silicon dioxide, acacia, agar, carrageenan, guar gum, locust bean gum, pectin, sodium alginate, propylene glycol alginate, tamarind gum, xanthan gum, carbomer, povidone, sodium starch glycolate, starches, tragacanth, magnesium aluminum silicate, aluminum silicate, magnesium silicate, gelatin, glycyrrhizin and the like. These suspending agents can further impart different flow properties to the suspension. The flow properties of the suspension can be Newtonian, plastic, pseudoplastic, thixotropic or combinations thereof. Mixtures of suspending agents may also be used to optimize flow properties and viscosity.

Moreover, the composition of the invention optionally include usual auxiliaries known in the art such as saliva stimulating agents like citric acid, lactic acid, malic acid, succinic acid, ascorbic acid, adipic acid, fumaric acid, tartaric acids; cooling sensation agents like maltitol, monomenthyl succinate, ultracool; stabilizers like gums, agar; taste masking agents like acrylic polymers, copolymers of acrylates, celluloses, resins; coloring agents like titanium dioxide, natural food colors, dyes suitable for food, drug and cosmetic applications; preservatives like alpha-tocopherol, citric acid, butylated hydroxytoluene, butylated hydroxyanisole, ascorbic acid, fumaric acid, malic acid, sodium ascorbate or ascorbic acid palmitate or effervescing agents like citric acid, tartaric acid, sodium bicarbonate, sodium carbonate and the like.

The formulations of the invention optionally include one or more stabilizing agents to increase the stability and/or compatibility of the suspension when formulated into a dosage form. Suitable stabilizing agents are suspending agents, flocculating agents, thickening agents, gelling agents, buffering agents, antioxidants, preservatives, antimicrobial agents, and mixtures thereof. Ideally, the agent acts to minimize irreversible aggregation of suspended particles, and to maintain proper flow characteristics to ease manufacturing processes, e.g., to ensure that the formulation can be readily pumped and filled into desired container.

The pharmaceutical composition of the present invention can be formulated by the various processes known in the art.

The invention is further illustrated by the following examples which are provided merely to be exemplary of the invention and do not limit the scope of the invention. Certain modifications and equivalents will be apparent to those skilled in the art and are intended to be included within the scope of the invention.

### EXAMPLE 1

### Oral Liquid Concentrate of Dextromethorphan Hydrobromide, Pseudoephedrine Hydrochloride, and Brompheniramine Maleate

TABLE 1

| Sr. No. | Ingredients | Quantity (% w/v) |
|---|---|---|
| 1 | Dextromethorphan Hydrobromide, | 0.25 |
| 2 | Pseudoephedrine Hydrochloride, | 0.75 |
| 3 | Brompheniramine Maleate, | 0.05 |
| 4 | Sorbitol 70% Solution, | 0.00 to 70.00 |
| 5 | Sodium Benzoate, | 0.05 to 0.15 |
| 6 | Methylparaben, | 0.05 to 0.20 |
| 7 | Citric Acid anhydrous, | 0.039 to 0.30 |
| 8 | Sodium Citrate dihyrdate, | 0.026 to 0.385 |
| 9 | Sucralose, | 0.1 to 0.50 |
| 10 | Glycerin, | 0 to 15.0 |
| 11 | Propylene Glycol, | 0.0 to 10.0 |
| 12 | FD & C Red No. 40 | 0.0003 to 0.007 |
| 13 | Artificial Butterscotch Flavor F-1785 | 0.10% to 0.20 |
| 14 | Purified water, | Q.s to 100 |

Procedure:

Purified water and glycerin were mixed in a manufacturing tank. Propylene Glycol was transferred to Jacketed SS tank and heated to 45° C-50° C. Methylparaben was added to warm Propylene glycol and dissolved by stirring. This solution of methylparaben was transferred to main manufacturing tank. Citric acid anhydrous, Sodium citrate dihydrate were added to the solution in main tank under stirring. Dextromethorphan hydrobromide, Brompheniramine maleate, Pseudoephedrine hydrochloride, Sodium benzoate, Sucralose and Sorbitol were added to the above solution and stirred till dissolved completely. Color solution prepared by dissolving color in water and Artificial Butterscotch flavor was added to above solution. The solution was filtered, and filled in amber HDPE bottle.

Stability Studies: The

composition of in Example 1 was subjected to stability study at accelerated stability conditions i.e. 40° C./25% Relative Humidity as well as at room temperature i.e. 25° C./40% Relative Humidity (RH). The samples were withdrawn initially, at 1 month, 2 months and 3 months and were analyzed using HPLC. The results obtained are reproduced below in Table 2.

TABLE 2

| | | | | Accel. stability | | | |
|---|---|---|---|---|---|---|---|
| | | | | studies | | | |
| Sr. | | | | 40° C./25% RH | | | 25° C./40% RH |
| No. | Test | Specification | Initial | 1M | 2M | 3M | 3M |
| 1 | Description | Clear light | Complies | Y | Y | Y | Y |
| | | pink Syrup | (Y) | | | | |
| | | having | | | | | |
| | | butterscotch | | | | | |
| | | flavor | | | | | |
| 2 | Specific gravity at | 1.128-1.246 | 1.187 | 1.187 | 1.187 | 1.187 | 1.187 |
| | 25° C. | | | | | | |
| 3 | pH | 3.5-5.0 | 4.52 | 4.49 | 4.47 | 4.46 | 4.45 |
| 4 | Viscosity | 1-50 cps | 11.73 | 11.73 | 11.73 | 11.73 | 11.73 |
| 5 | Assay | | | | | | |
| A) | Dextromethorphan | 90-110% | 99.10 | 98.70 | 99.30 | 101.10 | 100.50 |
| | Hydrobromide | | | | | | |
| B) | Pseudoephedrine | 90-110% | 100.10 | 98.50 | 98.90 | 101.50 | 100.90 |
| | Hydrochloride | | | | | | |
| C) | Brompheniramine | 90-110% | 100.10 | 99.20 | 99.20 | 102.70 | 101.70 |
| | Maleate | | | | | | |
| 6 | Related substances | | | | | | |
| i) | Pheniramine | 0.5% | 0.069 | 0.029 | 0.021 | 0.046 | 0.035 |
| | Maleate | | | | | | |
| ii) | Highest unknown | 0.2% | 0.013 | 0.028 | 0.049 | 0.116 | 0.027 |
| | Impurity | | | | | | |
| iii) | Total related | 1.5% | 0.148 | 0.341 | 0.361 | 0.388 | 0.211 |
| | Substances | | | | | | |

While the present invention has been described in terms of its specific embodiments, certain modifications and equivalents will be apparent to those skilled in the art and are intended to be included within the scope of the present invention.

We claim:

1. An oral ready-to-use pharmaceutical liquid composition comprising more than 0.04% w/v of brompheniramine, 0.60% w/v of pseudoephedrine and 0.20% w/v of dextromethorphan or pharmaceutically acceptable salts thereof.

2. The oral ready-to-use pharmaceutical liquid composition of claim 1, wherein the composition is free of alcohol.

3. The oral ready-to-use pharmaceutical liquid composition of claim 1, wherein the composition is free of sugar.

4. The oral ready-to-use pharmaceutical liquid composition of claim 1, wherein the composition is in the form of a liquid concentrate.

5. The oral ready-to-use pharmaceutical liquid composition of claim 1, wherein the composition comprises hydrobromide salt of dextromethorphan, maleate salt of brompheniramine, and hydrochloride salt of pseudoephedrine.

6. The oral ready-to-use pharmaceutical liquid composition of claim 1, wherein the composition is taste-masked.

7. The oral ready-to-use pharmaceutical liquid composition of claim 1, wherein the composition is in the form of solution, syrup, suspension, or emulsion.

8. The oral ready-to-use pharmaceutical liquid composition of claim 1, wherein the composition retains at least 90% w/w of total potency of brompheniramine, pseudoephedrine, and dextromethorphan or pharmaceutically acceptable salts thereof after storage at 25° C. and 40% relative humidity or 25° C. and 40% relative humidity for at least 3 months.

9. An oral ready-to-use pharmaceutical liquid concentrate comprising about 0.05% w/v of brompheniramine, 0.75% w/v of pseudoephedrine and 0.25% w/v of dextromethorphan or pharmaceutically acceptable salts thereof, wherein the composition is free of alcohol.

10. An oral ready-to-use pharmaceutical liquid composition comprising about 10 mg of dextromethorphan, about 2 mg of brompheniramine and about 30 mg of pseudoephedrine or pharmaceutically acceptable salts thereof in each 4 ml of the liquid composition along with one or more pharmaceutically acceptable excipients.

11. The oral ready-to-use pharmaceutical liquid composition of claim 10, wherein the composition is free of alcohol.

12. The oral ready-to-use pharmaceutical liquid composition of claim 10, wherein the composition is free of sugar.

13. The oral ready-to-use pharmaceutical liquid composition of claim 10, wherein the composition comprises hydrobromide salt of dextromethorphan, maleate salt of brompheniramine, and hydrochloride salt of pseudoephedrine.

14. The oral ready-to-use pharmaceutical liquid composition of claim 10, wherein the composition retains at least 90% w/w of total potency of brompheniramine, pseudoephedrine, and dextromethorphan or pharmaceutically acceptable salts thereof after storage at 25° C. and 40% relative humidity or 25° C. and 40% relative humidity for at least 3 months.

15. The oral ready-to-use pharmaceutical liquid composition of claim 10, wherein the composition is taste-masked.

16. A method of treating one or more symptoms selected from upper respiratory tract infection, common cold, and allergic rhinitis, which method comprises of administering the oral ready-to-use pharmaceutical liquid composition of claim 1 to a patient in need thereof.
